(19) United States
(12) Patent Application Publication
(10) Pub. No.: US 2006/0134656 A1
(43) Pub. Date: Jun. 22, 2006

(54) Reduction of non-specific adsorption of biological agents on surfaces

(76) Inventors: Robert J. Hamers, Madison, WI (US); Tami Lasseter Clare, Philadelphia, PA (US); Nicholas L. Abbott, Madison, WI (US); Brian H. Clare, Philadelphia, PA (US)

Correspondence Address: FOLEY & LARDNER LLP, 150 EAST GILMAN STREET, P.O. BOX 1497, MADISON, WI, 53701-1497, US

(21) Appl. No.: 11/137,127

(22) Filed: May 25, 2005

Related U.S. Application Data
(60) Provisional application No. 60/636,639, filed on Dec. 16, 2004.

(51) Int. Cl. C12Q 1/68; G01N 33/53; C12M 1/34

(52) U.S. Cl. 435/6; 435/7.1; 435/287.2; 435/7.5

(57) ABSTRACT

The present invention relates to surface-modified substrates that demonstrate reduced non-specific adsorption of biological agents. The substrates are silicon or carbon substrates having ethylene glycol oligomers covalently bound to at least one substrate surface. The substrates may be used in sensor devices, such as biochips, and in implantable medical devices in order to reduce the non-specific binding of biological agents.

### CROSS-REFERENCE TO RELATED PATENT APPLICATIONS

This application claims the benefit of U.S. provisional patent application No. 60/636,639, filed Dec. 16, 2004, the entire disclosure of which is incorporated herein by reference and for all purposes.

### STATEMENT OF GOVERNMENT RIGHTS

Research funding was provided for this invention by the National Science Foundation under grant Nos. NSF: 0314618 and 0079983. The United States government has certain rights in this invention.

### FIELD OF THE INVENTION

This invention relates to substrates that exhibit reduced non-specific binding of biological agents. More specifically, this invention relates to silicon and carbon substrates having a layer of ethylene glycol oligomers covalently bound to their surfaces.

### BACKGROUND OF THE INVENTION

Oligoethylene glycol monolayers on gold and SiO₂ surfaces have been used to resist the non-specific adsorption of proteins and cells. See Pale-Grosdemange, C.; Simon, E. S.; Prime, K. L.; Whitesides, G. M. J. Am. Chem. Soc. 1991, 113, 12-20. Ostum, E.; Yan, L.; Whitesides, G. M., Colloids Surf., B 1999, 15, 3-30; Sharma, S.; Johnson, R. W.; Desai, T. A. Langmuir 2004, 20, 348-356; and Faucheux, N.; Schweiss, R.; Lutzow, K.; Wemer, C; Groth, T. Biomaterials 2004, 25, 2721-2730. However, almost all previous studies of oligo(ethylene glycol)-modified surfaces have been performed on SAMs on silver and gold, linking oligo(ethylene glycol) alkanethiols to the surface by Ag—S or Au—S bonds. (See, for example, Prime, K. L.; Whitesides, G. M., Science 1991, 252, 1164-1167; Harder, P.; Grunze, M.; Dahint, R.; Whitesides, G. M.; Laibinis, P. E., J. Phys. Chem. B 1998, 102, 426-436; Pale-Grosdemange, C.; Simon, E. S.; Prime, K. L.; Whitesides, G. M., J. Am. Chem. Soc. 1991, 113, 12-20.) While conventional SAMs on gold and silver can optimize alkyl chain packing by lateral diffusion of the metal-thiol bonds, the covalent bonds of molecules to Si or diamond prevent any lateral movement of the molecules and leads to molecular layer that is not as well-packed. Recent studies have suggested that closely-spaced, crystalline-like monolayers are less resistant to non-specific adsorption than similar layers with structural or chemical disorder. (Harder, P.; Grunze, M.; Dahint, R.; Whitesides, G. M.; Laibinis, P. E., J. Phys. Chem. B 1998, 102, 426-436; Ostuni, E.; Yan, L.; Whitesides, G. M., Colloids and Surfaces B: Biointerfaces 1999, 15, 3-30; Li, L.; Chen, S.; Zheng, J.; Ratner, B. D.; Jiang, S., J. Phys. Chem. 2004, in press; Herrwerth, S.; Eck, W.; Reinhardt, S.; Grunze, M., J. Am. Chem. Soc. 2003, 125, 9359-9366; Zwahlen, M.; Herrwerth, S.; Eck, W.; Grunze, M.; Hahner, G., Langmuir 2003, 19, 9305-9310; Schwendel, D.; Dahint, R.; Herrwerth, S.; Schloerholz, M.; Eck, W.; Grunze, M., Langmuir 2001, 17, 5717-5720.)

Non-specific adsorption of proteins at surfaces leads to fouling of biosensors, decreased performance and failure of indwelling devices such as implants, stents, and electrodes, and decreased sensitivity of medical tests that detect binding of specific proteins. Thus, the ability to resist biofouling is important for the design of biocompatible coatings (e.g., diamond and diamond-like carbon) for implants and for biosensors capable of detecting analytes in complex protein mixtures.

Covalently modified surfaces of silicon and of diamond thin films are now emerging as useful materials for the direct electrical detection of biomolecules. See Lasseter, T. L.; Cai, W.; Harriers, R. J. Analyst 2004, 129, 3-8. Cai, W.; Peck, J. R.; van der Weide, D. W.; Harriers, R. J. Biosens. Bioelectron. 2004, 19, 1013-1019; and Yang, W. S.; Auciello, O.; Butler, J. E.; Cai, W.; Carlisle, J. A.; Gerbi, J.; Gruen, D. M.; Knickerbocker, T.; Lasseter, T. L.; Russell, J. N., Jr.; Smith, L. M.; Harriers, R. J. Nat. Mater. 2002, 1, 253-257. Recent studies have reported that monolayers on gold and SiO₂ can be unstable when used over the span of many days, while monolayers on silicon and carbon-based materials show promise for longer-term stability. See Flynn, N. T.; Tran, T. N. T.; Cima, M. J.; Langer, R. Langmuir 2003, 19, 10909-10915; Cai, W.; Peck, J. R.; van der Weide, D. W.; Harriers, R. J. Biosens. Bioelectron. 2004, 19, 1013-1019; Yang, W. S.; Auciello, O.; Butler, J. E.; Cai, W.; Carlisle, J. A.; Gerbi, J.; Gruen, D. M.; Knickerbocker, T.; Lasseter, T. L.; Russell, J. N., Jr.; Smith, L. M.; Harriers, R. J. Nat. Mater. 2002, 1, 253-257; and Buriak, J. Chem. Comm. 1999, 12, 1051-1060.

Covalent modification of Si(111) surfaces through Si—C bond formation can be achieved because vinyl groups will photochemically react directly with a surface, producing covalently linked monolayers that can serve as stable anchor points for tethering biological molecules to the surface. See Buriak, J. Chem. Comm. 1999, 12, 1051-1060; Cicero, R. L.; Linford, M. R.; Chidsey, C. E. D. Langmuir 2000, 16, 5688-5695; and Strother, T.; Harriers, R. J.; Smith, L. M. Nucleic Acids Res. 2000, 28, 3535-3541. Diamond surfaces can be modified similarly, producing DNA layers exhibiting higher stability than those on gold, silicon, and SiO₂. See Yang, W. S.; Auciello, O.; Butler, J. E.; Cai, W.; Carlisle, J. A.; Gerbi, J.; Gruen, D. M.; Knickerbocker, T.; Lasseter, T. L.; Russell, J. N., Jr.; Smith, L. M.; Harriers, R. J. Nat. Mater. 2002, 1, 253-257. However, methods for reducing non-specific binding on silicon and diamond surfaces have generally remained relatively unexplored. See Zhu, X. Y.; Jun, Y.; Staarup, D. R.; Major, R. C.; Danielson, S.; Bioadjiev, V.; Gladfelter, W. L.; Bunker, B C.; Guo, A. Langmuir 2001, 17, 7798-7803.

### SUMMARY OF THE INVENTION

The present invention relates to surface-modified substrates that demonstrate reduced non-specific adsorption of biological agents. The substrates are silicon or carbon substrates having ethylene glycol oligomers covalently bound to at least one substrate surface. The substrates may be used in sensor devices, such as biochips, and in implantable medical devices in order to reduce the non-specific binding of biological agents.

In one embodiment, the surface-modified substrate is a silicon or carbon substrate having a layer of ethylene glycol oligomers covalently bound thereto. In another embodiment, the surface-modified substrate is a silicon or carbon substrate having a mixed layer of ethylene glycol oligomers and probe molecules covalently bound thereto. The probe molecules may be any biomolecule capable of undergoing a specific binding interaction with a target molecule of interest. By exposing the surface-modified substrate to an analyte sample, the presence of target molecules in the sample may be confirmed by detecting target molecules that have undergone specific binding with the surface-bound probe molecules. Because the ethylene glycol oligomers reduce non-specific binding between the target molecules and the surface, sensors made from the present surface-modified substrates are more sensitive than other similar biosensors.

The ethylene glycol oligomers used to modify the surfaces include a terminal vinyl group that reacts with the substrate surface to form a covalent bond. The ethylene glycol oligomers may be represented by the formula: CH₂═CH(CH₂)ₘ(OCH₂CH₂)ₙOR, where m>0, n>2 and R represents a terminal functional group or atom. Useful ethylene glycol oligomers include those where 0<m≧20, 3≦n≧20 and R is an H atom or a methyl group.

Further objects, features and advantages of the invention will be apparent from the following detailed description when taken in conjunction with the accompanying drawings.

### BRIEF DESCRIPTION OF THE DRAWINGS

FIG. 1 shows the fluorescence intensity as a function of percentage Boc-N-ene in mixed monolayers of EG3-ene and Boc-N-ene on silicon (a), gold (b) and diamond (c) surfaces.

FIG. 2 is a schematic diagram of avidin target molecules specifically and non-specifically bound to biotin probe molecules on a silicon substrate having a mixed monolayer covalently bound to its surface.

FIG. 3 is a plot of the ratio of specific to non-specific binding of avidin molecules to a biotin molecules bound to a mixed monolayer on a silicon surface, as a function of the percentage of Boc-N-ene in the monolayer.

FIG. 4 is a schematic diagram of a method for making a surface-modified silicon or diamond substrate in accordance with the present invention.

FIG. 5 is a schematic diagram of surfaces modified with layers of EG3-ene molecules, amino-terminated linking molecules, EG6-ene molecules, and Me-EG3-ene molecules.

FIG. 6 shows a plot of fluorescence intensity of fluorescently-labeled proteins on a surface-modified diamond substrate as a function of percentage amino-terminated linker molecule on the surface (left panel) and as a function of ethylene glycol oligomer chain length (right panel).

FIG. 7 shows a plot of fluorescence intensity of fluorescently-labeled proteins on a surface-modified silicon substrate as a function of percentage amino-terminated linker molecule on the surface (left panel) and as a function of ethylene glycol oligomer chain length (right panel).

FIG. 8 shows a plot of fluorescence intensity of fluorescently-labeled proteins on a surface-modified silicon substrate as a function of percentage Me-EG3-ene and EG3-ene on the surface.

FIG. 9 is a schematic diagram showing a reaction scheme for covalently bonding a biotin molecule to a substrate using an amino-terminated linking molecule and for detecting avidin molecules specifically bound to the biotin.

FIG. 10 shows a plot of adsorption of avidin (in percent monolayer equivalents) on biotinylated, amino-terminated, EG3-ene functionalized and EG6-ene functionalized substrates of diamond, silicon and gold.

FIG. 11 is a schematic diagram of avidin specifically binding to biotinylated silicon and non-specifically adsorbing onto (a) 100% amino-terminated and (b) 90% EG6, 10% amino-terminated monolayers on silicon.

FIG. 12 is a plot showing the optimization of specific (S) and nonspecific (NS) binding of avidin to silicon covalently modified from mixed solutions of EG6-ene/Boc-N-ene (diamond shapes) and EG3-ene/Boc-N-ene (squares). The double dagger, ‡, indicates data from FIG. 11(a) and the asterisk, *, indicates data from FIG. 11(b).

FIG. 13 is a plot showing percent EG moiety on surface (from XPS measurements) versus percent EG moiety in parent solution. Points A, B, C, D, and E are discussed in the text.

FIG. 14 is a table providing some numerical data from XPS spectra of points A-E of FIG. 13.

FIG. 15 is a plot showing a comparison of mixed biotinylated/EG6 monolayers and 100% biotinylated monolayers on silicon for their ability to detect fluorescein-labeled avidin in undiluted chicken serum.

### DETAILED DESCRIPTION OF THE INVENTION

The present invention utilizes the direct covalent functionalization of silicon and carbon substrates with short ethylene glycol (EG) oligomers via photochemical reaction of the hydrogen-terminated surfaces with terminal vinyl groups of the oligomers. The functionalized surfaces effectively resist the non-specific adsorption of proteins and other biological agents. Mixed monolayers can be prepared on silicon and carbon and these surfaces can be applied to optimize the ratio of specific to non-specific binding in a model biomolecule sensing assay.

Substrates to which the EG oligomers may be bound in accordance with the present invention include silicon and carbon substrates. Single crystal silicon substrates having the EG oligomers bound to the Si(111) surface are one specific example of a suitable silicon substrate. Examples of suitable carbon substrates include, but are not limited to, substrates composed of diamond, diamond-like carbon, glassy carbon, graphitic carbon and pyrolytic carbon. In some instances, the carbon material may be deposited as a layer over an underlying support, as in the case of a diamond-like carbon film. It should be understood that in these cases the term “substrate” would refer to the carbon layer and not to the underlying support. As one of skill in the art would understand, diamond-like carbon films are hard, carbon films with a significant fraction of sp3-hybridized carbon atoms. These film may contain a significant amount of hydrogen, or may be produced with little or no hydrogen. Depending on the deposition conditions, the diamond-like carbon films can be fully amorphous or contain diamond crystallites. In some embodiments, the diamond-like carbon films may be nanocrystalline films. In still other embodiments, the carbon substrate may be composed of carbon nanoparticles, such as carbon nanotubes or Buckyballs.

The EG oligomers used to make the surface-modified substrates include a terminal vinyl group for reacting with the silicon or carbon surface. The oligomers are generally represented by the following formula: CH₂═CH(CH₂)ₘ(OCH₂CH₂)ₙOR, where m is greater than or equal to 1 and n is at least 3 and R is a terminal functional group or atom. In some exemplary embodiments, m has a value from 1 to 20. This includes embodiments where m has a value from 1 to 12 and further includes embodiments where m has a value from 3 to 10. In some exemplary embodiments, n has a value from 3 to 15. This includes embodiments where n has a value from 3 to 12 and further includes embodiments where n has a value from 3 to 9. Specific examples of suitable EG oligomers that may be used to modify silicon and carbon substrate surfaces include, but are not limited to, triethylene glycol undec-1-ene, monomethyl triethylene glycol undec-1-ene, tetraethylene glycol undec-1-ene, pentaethylene glycol undec-1-ene and hexaethylene glycol undec-1-ene.

Unlike polyethylene glycol polymers, the ethylene glycol oligomers generally have dimensions shorter than the dimensions of proteins and have a defined terminal tether point where their vinyl group has reacted with the substrate surface. As a result, the ethylene glycol oligomers form oriented structures which differ from polyethylene glycol polymer coatings which are relatively thick and which bind to a surface at many points along the backbones of the polymer chains. It should be noted, however, that although the ethylene glycol oligomers are bound primarily through the vinyl group, some of the of the oligomers may bind through other functionalities, such as a terminal hydroxyl group. This may lead to some chemical and structural disorder in the layer. Thus, structural perfection of the layer is not necessary in order to resist non-specific adsorption, and indeed, some disorder may even be beneficial.

The terminal group (R) on the free end of the surface-bound EG oligomers may be any functional group that provides a modified surface exhibiting reduced non-specific adsorption of biological agents. For example, R may be an H atom, an alkyl group, an amino group or a carboxylic acid group. In some embodiments R is a methyl group. However, the inventors have surprisingly discovered that in some embodiments it is preferable for R to be an H atom, such that the EG oligomers are terminated by hydroxyl groups, because the hydroxyl-terminated EG oligomer layers may provide improved resistance to non-specific binding of biological agents. This contravenes recent thinking on this issue wherein it has been proposed that methyl-terminated EG monolayers should be more useful than hydroxyl-terminated monolayers for many in vivo applications because the methyl group cannot be oxidized. (See, for example, Ostuni, E.; Chapman, R. G.; Liang, M. N.; Meluleni, G.; Pier, G.; Ingber, D. E.; Whitesides, G. M., Langmuir 2001, 17, 6336-6343; Faucheux, N.; Schweiss, R.; Lutzow, K.; Werner, C.; Groth, T., Biomaterials 2004, 25, 2721-2730.) The present inventors have discovered that although hydroxyl groups may be oxidized, they may be more effective than terminal-methyl groups at resisting protein adsorption.

The EG oligomers desirably form a layer, which is preferably a monolayer, on at least a portion of a silicon or carbon substrate surface. The layer may be a pure or substantially pure EG oligomer layer wherein the only molecules covalently bound to the surface are EG oligomers. Alternatively, the layer may be a mixed layer containing a mixture of EG oligomers and probe and/or linking molecules covalently bound to the surface. The latter design is particularly useful in the production of sensing devices. In this design, the probe molecules in the layer are capable of undergoing specific binding to target molecules in a sample while the EG oligomers in the layer reduce non-specific binding of the target molecules to the substrate. The ratio of EG oligomers to probe molecules may be tailored to maximize the specific to non-specific binding ratio for the sensor.

In some instances the probe molecules will themselves include functional groups capable of reacting with and bonding to the substrate surface. More commonly, however, the probe molecules will be composed of molecules functionalized with a functional group that provides reactivity and bonding between the probe molecule and a linking molecule. In this construction the linking molecules are covalently bound to both a probe molecule and the substrate, such that the linking molecules provide tethers anchoring the probe molecules to the substrate. The linking molecules may serve to properly orient the probe molecule for interaction with the target molecules. Additionally, in cases where the probe molecules are bioactive biomolecules, such as enzymes, the linking molecules may be used to optimize the spacing between the substrates and the probe molecules so that the biomolecules retain their bioactivities.

The probe molecules may be any molecules that undergo a specific binding interaction with one or more target molecules in a sample. Suitable probe molecules include, but are not limited to, biomolecules selected from the group consisting of oligonucleotide sequences, including both DNA and RNA sequences, amino acid sequences, proteins, protein fragments, ligands, receptors, receptor fragments, antibodies, antibody fragments, antigens, antigen fragments, enzymes, enzyme fragments and combinations thereof. Thus, the specific binding interactions between the probe and target molecules include, but are not limited to, receptor-ligand interactions (including protein-ligand interactions), hybridization between complementary oligonucleotide sequences (e.g. DNA-DNA interactions or DNA-RNA interactions), and antibody-antigen interactions. (For the purposes of this disclosure, the terms “specific adsorption” and “specific binding” are used interchangeably.) In one exemplary embodiment of the invention the target molecules are proteins and the probe molecules are ligands capable of specifically binding with the proteins. For example, the protein may be avidin or Streptavidin and the ligand may be biotin.

The linking molecules may be any molecules capable of covalently bonding to the substrate and to a probe molecule to tether the probe molecule to the surface of the substrate. Examples of useful linker molecule functionalities that may engage in covalent bonding with the substrate surface or a probe molecules include, but are not limited to, amino groups, epoxy groups, aldehyde groups, carboxyl groups, mercapto groups, chloracid groups and ester groups. Linking molecules having amino functionalities may be particularly useful because reactions between primary amino groups and a variety of other functional groups are known. For example, descriptions of reaction schemes for immobilizing biomolecules, such as DNA molecules, antibodies and nanostructures, on amino terminated substrates, including diamond and glassy carbon substrates may be found in Yang et al., Nature Materials, 1, 253-257 (2002); Strother et al., J.A.C.S., 122, 1205-1209 (2000); and Baker et al., Science, 293, 1289-1292 (2001), the entire disclosures of which are incorporated herein by reference.

The ratio or EG oligomers to probe or linking molecules in a mixed layer may be optimized to maximize the ratio of specific to non-specific binding of target molecules to the surface-modified substrate. In some instances the ratio of specific to non-specific binding of target molecules, such as biomolecules (e.g., proteins), may be optimized by using a layer comprising about 60 to 80% EG oligomers and about 20 to 40% probe molecules. This includes embodiments wherein the layer contains about 65 to 75% EG oligomers and about 25 to 35% probe molecules and further includes embodiments wherein the layer contains about 68 to 72% EG oligomers and about 28 to 32% probe molecules.

The EG oligomers and probe and/or linking molecules in a mixed layer are randomly distributed within the layer, although the layer itself may be patterned on the substrate. Thus, the present mixed layers would be distinguishable from an EG oligomer layer wherein some oligomers are selectively removed from a selected location in the layer and replaced by probe molecules.

The surface-modified substrates may be made by exposing hydrogen-terminated silicon or carbon surfaces to a parent liquid containing EG oligomers under ultraviolet (UV) light for a time sufficient to allow for the photochemical reaction of the EG oligomers with the substrate surface. In the case of mixed monolayers, the parent liquid may also contain linking molecules. For example, the parent liquid may include a mixture of EG oligomers and protected amino-functional linking molecules. The surface-bound linking molecules may then be deprotected and reacted with probe molecules. A more detailed description of methods for fabricating the surface-modified substrates may be found in the Examples section below.

The surface-modified substrates having a uniform layer of EG oligomers covalently bound thereto are useful in the fabrication of implantable medical devices because they reduce biofouling. Implantable medical devices that may benefit from surface modification with EG oligomers include, but are not limited to, prostheses, bone screws and hardware, surgical instruments, artificial organs, pacemakers and dental appliances.

The surface-modified substrates having a mixed layer of EG oligomers and probe molecules covalently bound thereto are useful in the fabrication of sensors, including biosensors (e.g., biochips). In these devices the mixed layer may be a discontinuous layer forming an array of islands on the substrate. Alternatively, the layer may be a continuous layer wherein the probe molecules are bound to the layer in an array of islands separated by sections of the layer that contain EG oligomers and linking molecules that have not been reacted with probe molecules. Examples of sensor devices that use biotin probe molecules are presented in the Examples section which follows.

### EXAMPLES

### Example 1

### Mixed Monolayers of Triethylene Glycol Oligomers and Amine-Functional Molecules on Silicon and Diamond Surfaces

Mixed monolayers presenting both amine and triethylene glycol (EG3) functionalities were prepared on silicon and diamond substrates. The incorporation of amines into the monolayer allowed for subsequent chemical modification of these interfaces. The mixed monolayers were formed by applying solutions of various mole percentages of triethylene glycol undec-1-ene (EG3-ene) and t-Boc 10-aminodec-1-ene (BocN-ene) onto hydrogen-terminated silicon (111) surfaces or TFA protected 10-aminodec-1-ene (TFA-N-ene) onto hydrogen-terminated polycrystalline, p-type diamond thin films. Methods for covalently attaching Boc-N-ene to silicon surfaces is described in Strother T; Hamers R. J.; Smith L. M.; NUCLEIC ACIDS RESEARCH 28 (18): 3535-3541 Sep. 15 2000. Methods for covalently attaching TFA-N-ene to diamond surfaces is described in Yang, W. S.; Auciello, O.; Butler, J. E.; Cai, W.; Carlisle, J. A.; Gerbi, J.; Gruen, D. M.; Knickerbocker, T.; Lasseter, T. L.; Russell, J. N., Jr.; Smith, L. M.; Harriers, R. J. Nat. Mater. 2002, 1, 253-257, the entire disclosure of which is incorporated herein by reference. Deposition of the liquids onto the surfaces followed by UV illumination at 254 nm for 3 hours (silicon) or 12 hours (diamond) linked the molecules to the surface via the vinyl group. Single-crystal and polycrystalline diamond samples showed nearly identical reactivity, indicating that defects and grain boundaries do not control the reaction of the polycrystalline films. Finally, the amino group was generated by the deprotection of the Boc or TFA group under acidic conditions. For comparison with previous studies, mixed monolayers were formed of amino-terminated and E133-terminated alkanethiols on gold. Methods for forming monolayers on gold are described in Prime, K. L.; Whitesides, G. M. Science 1991, 252, 1164-1167, and in Ostum, E.; Grzybowski, B. A.; Mrksich, M.; Roberts, C. S.; Whitesides, G. M. Langmuir 2003, 19, 1861-1872. Briefly, clean Au surfaces were immersed in 2 millimolar (mM) mixed solutions of 11-amino undecanethiol (Dojindo) and triethylene glycol undecanethiol (Prochimia) for at least 12 hours (h).

The monolayers were characterized using X-ray photoelectron spectroscopy (XPS) and the areas of the N(1 s) peak and the high binding energy C(1 s) peak at 287.2 eV were used to calculate the percentages of Boc-N-ene and EG3-ene in the mixed monolayers on silicon. Competitive binding experiments showed that, although the OH group and the vinyl group of the EG3-ene both can react with silicon, the vinyl group reacts approximately 3 times faster, so that ˜75% of EG3-ene molecules were bonded via the vinyl group, and 25% via the terminal O atom. At high amino concentrations the surface and solution compositions differed slightly as shown in Table 1. This difference likely arises from steric effects associated with the bulky t-Boc protecting group on the amine.

TABLE 1
Composition of the Mixed Monolayers on Silicon, Based upon
the Areas of the N(1s) and 287.2 eV C(1s) XPS Peaks

| mol % amino in liquid | mol % amino by XPS |
|---|---|
| 78 | 56 |
| 54 | 42 |
| 28 | 31 |

Fluorescence imaging was used to study the binding of fluorescently tagged avidin, bovine serum albumin (BSA), casein, and fibrinogen to these surfaces. High protein concentrations (0.2 mg/mL in 0.1 M NaHCO₃, pH 8.3), long binding times (1 h), and short rinsing times (1×15 min 2×SSPE buffer (Promega)+1% Triton-X 100) were chosen to challenge the resistance to non-specific binding. Fluorescence intensities were measured at 512 nm for fluorescein-labeled avidin, BSA, and casein, and at 550 mu for AlexaFluor546-conjugated fibrinogen using a Genomic Solutions UC4×4 fluorescence scanner. No significant lateral variations in intensity were detectable, indicating that adsorption occurred uniformly on optical length scales. The fluorescence intensities cannot be used to directly compare the absolute amount of non-specific binding on the different substrates because of differing amounts of fluorescence quenching. The fluorescence intensities were normalized to those of the 100% amino-terminated monolayers.

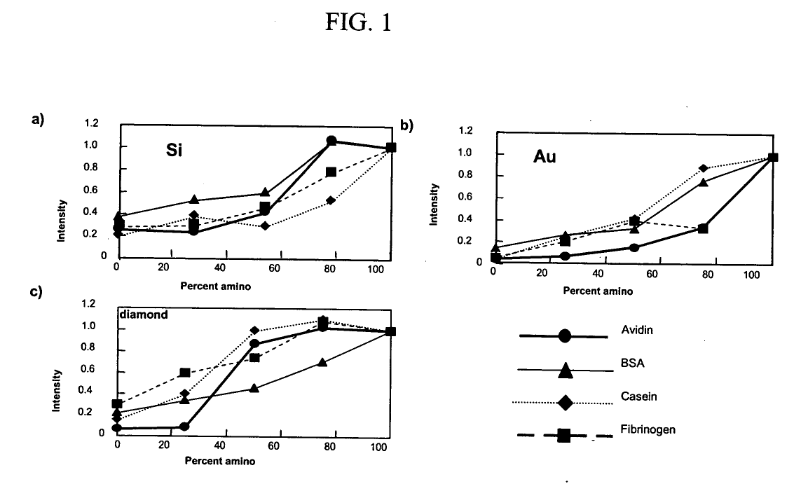

FIG. 1 shows plots of fluorescence intensity as a function of the percentage of Boc-N-ene in the mixed monolayer. The plots in FIG. 1 show that the EG3-ene oligomers on silicon (FIG. 1a), gold (FIG. 1b), and diamond (FIG. 1c) efficiently reduce non-specific adsorption of all of the proteins studied. The non-specific adsorption can be reduced by at least 60% on silicon, by 70% on diamond, and by 90% on gold surfaces.

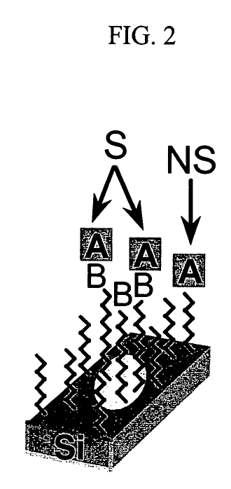

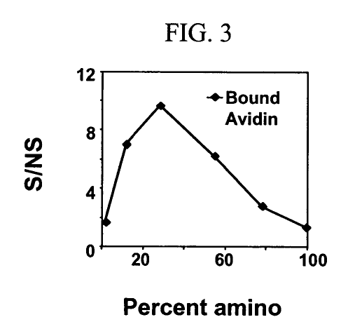

The properties of these new interfaces were exploited in the optimization of a standard protein assay. Utilizing the reactivity of the deprotected amino groups in mixed monolayers, biotin (the probe molecule) was incorporated into the interface using the amine-reactive biotin linker, sulfosuccininudyl-6′-(biotinamido)-6-hexaniido hexanoate (Pierce Endogen) (the linking molecule). Avidin (the target molecule) was allowed to bind to the entire surface for 10 min at 4° C., and the surface was briefly rinsed and then soaked for 15 min in 2×SSPE buffer +1% Triton-X 100. This process is described in greater detail in Lasseter, T. L.; Cai, W ; Harriers, R. J. Analyst 2004, 129, 3-8, the entire disclosure of which is incorporated herein by reference. An illustration of a surface-modified silicon substrate having biotin probe molecules (B) bound thereto and avidin target molecules (A) adsorbed thereon is provided in FIG. 2. Because EG3 functionalities reduce the amount of non-specific binding to the surface, the ratio of specifically bound avidin (the avidin that is retained on the biotinylated spot, S) to non-specifically bound avidin (the avidin that is retained on the rest of the monolayer, NS), S/NS, can be improved by using mixed monolayers containing EG3 functionalities as shown in FIG. 3. The improvement in SINS by forming mixed monolayers containing EG3 functionalities is a factor of 8, which was obtained using approximately 30% Boc-N-ene and 70% EG3-ene.

These results show that mixed monolayers containing EG3 functionality on silicon and diamond largely resist the non-specific adsorption of proteins. The highest S/NS was achieved using a mixed monolayer that allowed for specific binding while reducing non-specific binding. While previous work has shown that EG oligomers can reduce non-specific binding on gold, in many applications covalently functionalized materials such as silicon or diamond are advantageous because of their stability under a wide range of chemical and electrochemical conditions and because semiconductors provide a pathway for direct electrical sensing via field-effect devices. See Yang, W. S.; Auciello, O.; Butler, J. E.; Cai, W.; Carlisle, J. A.; Gerbi, J.; Gruen, D. M.; Knickerbocker, T.; Lasseter, T. L.; Russell, J. N., Jr.; Smith, L. M.; Harriers, R. J. Nat. Mater. 2002, 1, 253-257; and Prime, K. L.; Whitesides, G. M. Science 1991, 252, 1164-1167. The present invention thus provides a method for minimizing non-specific binding that can significantly enhance the ability to integrate biological molecules, especially proteins, with microelectronic materials.

### Example 2

### Mixed Monolayers of Triethylene Glycol Oligomers and Amine-Functional Molecules on Silicon and Diamond Surfaces

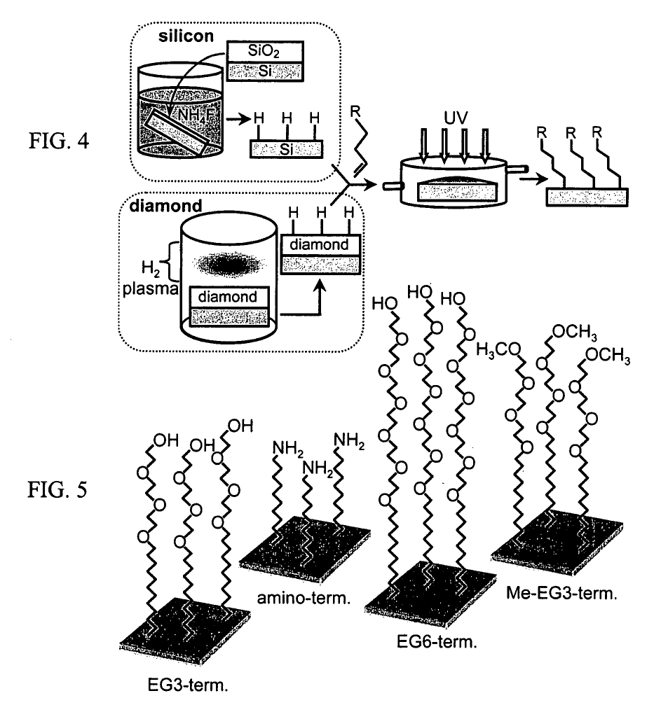

Hydrogen-terminated Silicon (111) surfaces were prepared by cleaning in acidic and basic solutions, followed by etching in nitrogen-sparged 40% NH₄F for 30 min. This process is described in greater detail in Strother, T.; Cai, W.; Zhao, X.; Hamers, R. J.; Smith, L. M., J. Am. Chem. Soc. 2000, 122, 1205-1209, the entire disclosure of which is incorporated herein by reference. Hydrogen-terminated diamond surfaces were prepared by acid cleaning followed by hydrogen plasma treatment, as reported in Strother, T.; Knickerbocker, T.; Russell, J. N. Jr.; Butler, J. E.; Smith, L. M.; Hamers, R. J., Langmuir 2002, 18, 968-971., the entire disclosure of which is incorporated herein by reference. Covalent monolayers were then formed on these surfaces by exposing the hydrogen-terminated surface to a parent liquid of the desired molecule under UV light for 3 h in the case of silicon, or 12 h in the case of diamond. To link amino groups to the surface, t-BOC 10 aminodec-1-ene (Boc-N-ene) and TFA-10 aminodec-1-ene (TFA-N-ene) were synthesized, covalently attached to silicon or diamond surfaces, respectively, and deprotected after attachment (and before characterization by XPS) as reported in Yang, W. S.; Auciello, O.; Butler, J. E.; Cai, W.; Carlisle, J. A.; Gerbi, J.; Gruen, D. M.; Knickerbocker, T.; Lasseter, T. L.; Russell, J. N., Jr.; Smith, L. M.; Hamers, R. J., Nature Materials 2002, 1, 253-257; Strother, T.; Hamers, R. J.; Smith, L. M., Nucleic Acids Research 2000, 28, 3535-3541; Strother, T.; Knickerbocker, T.; Russell, J. N. Jr.; Butler, J. E.; Smith, L. M.; Hamers, R. J., Langmuir 2002, 18, 968-971, the entire disclosures of which are incorporated herein by reference. Resistance to non-specific adsorption was conferred by binding vinyl-terminated ethylene glycol oligomer monolayers to the surface. Triethylene glycol-(EG3-ene), tetraethylene glycol-(EG4-ene), pentaethylene glycol-(EG5-ene), hexaethylene glycol-(EG6-ene), and monomethyl triethylene glycol-(Me-EG3-ene) undec-1-ene, were synthesized and fully characterized for these studies according to the procedures described in Pale-Grosdemange, C.; Simon, E. S.; Prime, K. L.; Whitesides, G. M., J. Am. Chem. Soc. 1991, 113, 12-20, the entire disclosure of which is incorporated herein by reference. A schematic diagram showing the process of forming the monolayers on silicon and diamond substrates is shown in FIG. 4. In this figure, the ethylene glycol oligomers are generically represented by a [symbol] R structure for simplicity. Illustrations of monolayers formed from these molecules are presented in FIG. 5. Monomethyl triethylene glycol (EG3-Me) and dimethyl triethylene glycol (Me-EG3-Me) were purchased from Aldrich. The various mixed monolayers were formed by making parent solutions of different compositions.

Preparation of Mixed Monolayers on Gold Surfaces. 100 nm Au films sputtered onto glass surfaces (GenTel) were cleaned for 15 minutes using a low-pressure mercury vapor quartz grid lamp, which removes adsorbed organic material on the gold surfaces. XPS measurements of these gold films (not shown) revealed a clean, carbon-free surface with only a trace of oxygen. The surfaces were then rinsed with H₂O followed by ethanol. The clean gold surfaces were immersed for at least 12 hours in 2 mM thiol solutions of: dodecanethiol (Dojindo), 11-aminoundecanethiol, MUAM (Dojindo), or triethylene glycol undecanethiol, EG3-SH (Prochimia).

Protein Adsorption. Fluorescein-labeled Casein (Sigma), fluorescein-labeled avidin (Vector Labs), fluorescein-labeled bovine serum albumin or BSA (Biømeda) and Fibrinogen Alexa Fluor 546 conjugate (Molecular Probes) were diluted or dissolved in 0.1 M NaHCO₃, pH 8.3, to a working concentration of 0.2 mg/mL. To test for non-specific adsorption, the proteins were spotted onto silicon or diamond surfaces on which a mixed or one component monolayer had been formed, allowed to adsorb at room temperature for one hour (the samples were kept in a humidified chamber during that time), briefly rinsed and then soaked for 15 minutes in 2×SSPE buffer (Promega)+1% Triton-X 100, the wash-off buffer. These adsorption reactions were characterized by on-chip fluorescence imaging (where the intensity of the adsorbed proteins on the surfaces was measured) or solution-based measurements (where adsorbed protein was eluted off of the surfaces and the intensity of fluorescence from the eluent was measured using a fluorometer.) For the latter method, the samples were soaked in 1.00 mL of the 2×SSPE buffer (Promega)+1% Triton-X 100+1% mercaptoethanol, the elution buffer, for at least 12 h. Mercaptoethanol is a reducing agent which acts to cleave disulfide bonds in proteins which aided their elution from the substrates into the elution buffer. The effectiveness of removal was checked by ensuring that little or no fluorescence remained on the surfaces after elution; the fluorescence intensity of the eluent containing the protein was then measured.

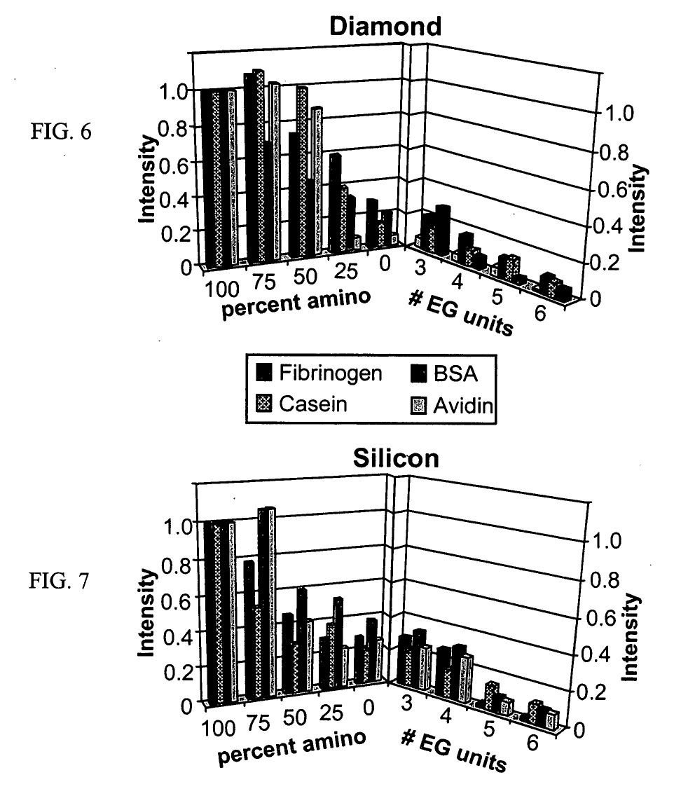

Fluorescence measurements. For the on-chip fluorescence measurements (FIGS. 6, 7, 8 and 12), the fluorescence intensity of the fluorescein-labeled proteins was measured using a Genomic Systems UC 4×4 fluorimager using a 488 nm excitation source and a 512 nm band pass filter, and the intensity of the Alexa Fluor 546 conjugated fibrinogen was measured using a 532 nm excitation source and a 550 nm long pass filter. In the solution-based method, fluorescence measurements of proteins collected in the elution buffer were performed using an ISS photon counting spectrofluorometer. Measurements of fluorescein-avidin were made by exciting at 480 nm and collecting the emission intensity at 518 nm; 1 mm slits, which act as 8 nm bandpass filters, were used.

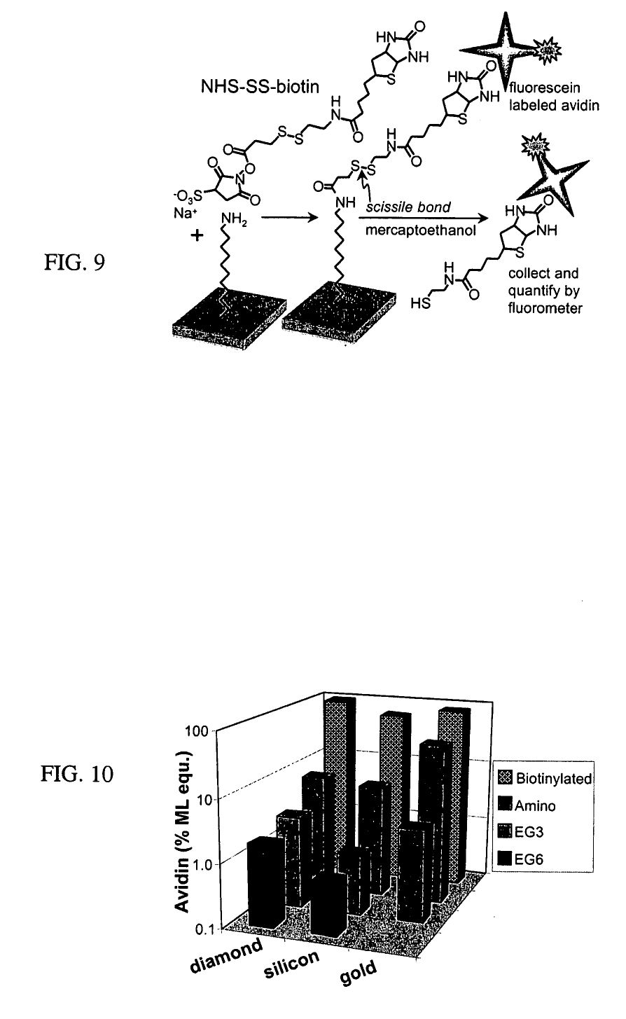

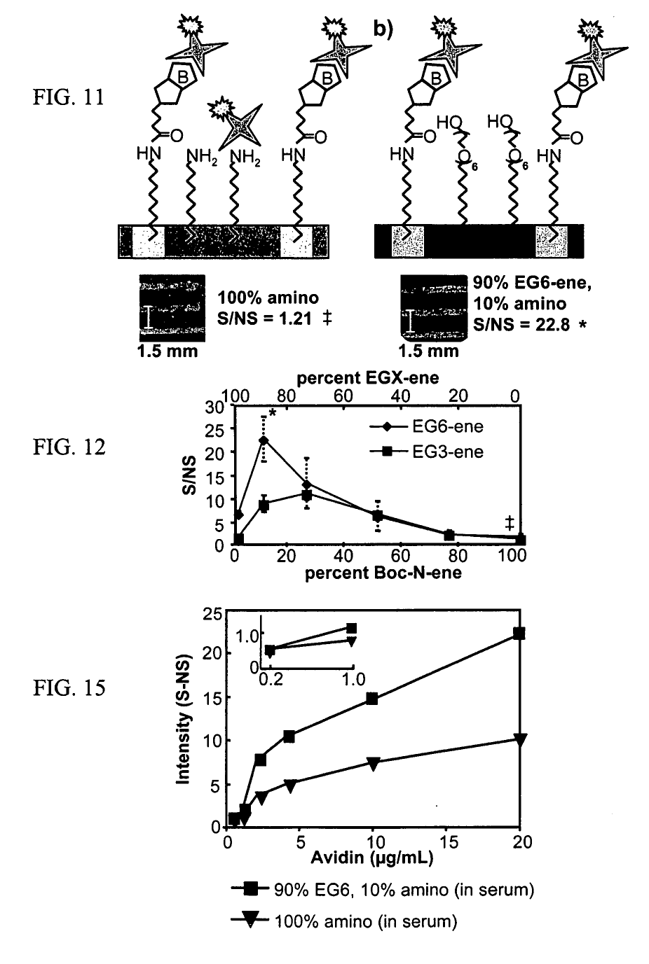

Specific Binding. The silicon surfaces were biotinylated by spotting a biotin linker, sulfo-succinimidyl-6′-(biotinamido)-6-hexamido hexanoate (Pierce Endogen) onto amino-terminated silicon surfaces as reported in Lasseter, T. L.; Cai, W.; Hamers, R. J., Analyst 2004, 129, 3-8, the entire disclosure of which is incorporated herein by reference. Avidin diluted (in the bicarbonate buffer as above) to a working concentration of 0.2 mg/mL was spotted onto biotinylated silicon surfaces, allowed to bind for 10 minutes at 4° C., briefly rinsed, and then soaked for 15 minutes in wash-off buffer. FIGS. 9 and 11 provide schematic diagrams of biotinylated substrates. Controls for specific binding, where biotin-saturated avidin in solution was exposed to biotinylated surfaces, showed no fluorescence intensity. Fluorescence intensities were immediately measured as described above. Competitive binding studies were performed using chicken serum purchased from Sigma.

XPS Characterization. Molecular layers on silicon were characterized using X-ray photoelectron spectroscopy, using a system equipped with a monochromatized Al K_{α} source and a multichannel array detector. Spectra reported here were recorded with an analyzer resolution of 0.18 eV. The percent EG moiety on the surface was calculated by fitting the carbon spectrum to two peaks and the nitrogen spectrum to one peak. The percent EG moiety was calculated from XPS data using the following equation: X=% EG moiety, 100−X=% Boc-N-ene (100−X)/(X)=[(low BE Carbon area)/(high BE Carbon area−Nitrogen area)]*(# C having high BE)/(# C having low BE). The nitrogen area was corrected for the sensitivity factor difference between nitrogen and carbon.

Results

On-chip fluorescence measurements were used to investigate qualitative trends in the reduction of non-specific adsorption as a function of monolayer composition. On-chip fluorescence intensities cannot be quantitatively compared between substrate types (i.e., silicon versus diamond) due to substrate-dependent fluorescence quenching. More quantitative measurements for comparison of adsorption on different substrates were made by eluting adsorbed avidin and measuring the fluorescence of the eluent as described above.

Effect of EG Chain Length on Protein Adsorption

This part of the example demonstrates how increasing the length of the EG chain can affect non-specific protein adsorption. In these studies, fluorescently labeled proteins were allowed to adsorb to functionalized silicon or nanocrystalline (NC) diamond, and the protein remaining was measured using on-chip fluorescence imaging. Illustrated in FIG. 4 is the reaction scheme for the chemical modification of silicon and diamond, and in FIG. 2 are the covalently bound monolayers that result when the hydrogen-terminated surfaces were exposed to Boc-N-ene (silicon) or to TFA-N-ene (diamond) and then deprotected, to EG3-ene, to EG6-ene, or to Me-EG3-ene.

Measurements of the fluorescence intensity after the fluorescently labeled proteins (avidin, BSA, casein, and fibrinogen) were adsorbed to separate areas of the functionalized surfaces and rinsed (as described above are) are shown in FIGS. 6 (diamond) and 7 (silicon). The data presented in FIGS. 6 and 7 were normalized to the amino-terminated surfaces in order to highlight the dramatic reduction of non-specifically adsorbed protein that occurs when EG units were incorporated into the monolayer. The left panels show the fluorescence intensity due to non-specific adsorption of proteins onto mixed monolayers of Boc-N-ene and EG3-ene on silicon and diamond, while the right panels show the effect of increasing EG chain length for pure EG monolayers.

The data in the left panels of FIGS. 6 and 7 show that the fluorescence intensity arising from each of the four proteins investigated decreases as more EG3 functionality is incorporated into the monolayers. The 100% EG3-functional monolayer yields a reduction in fluorescence intensity by as much as 60% (silicon) and 70% (diamond) compared with the amino-terminated surfaces; if the fluorescence intensity is assumed to be proportional to surface concentration, then this corresponds to a 60-70% reduction in non-specific adsorption. Repeated experiments showed a variation in fluorescence intensity of approximately 25% for each data point in FIGS. 6 and 7; thus, the slight difference between diamond and silicon is not significant. These results show that EG3-functional monolayers effectively reduce non-specific adsorption on both silicon and diamond surfaces.

The data in the right panels of FIGS. 6 and 7 show how the fluorescence intensity from adsorbed proteins varies as the EG chain increased from three to six EG units. These data illustrate that although EG3 functionality is effective at reducing non-specific adsorption, the amount of adsorbed protein can be further reduced by increasing the number of EG units in the oligomer. For example, the EG6 molecule yields an additional reduction of 50-90% on silicon and 50-80% on diamond compared with EG3, varying somewhat between different proteins.

Effect of Methyl-Terminated EG Monolayers on Protein Adsorption

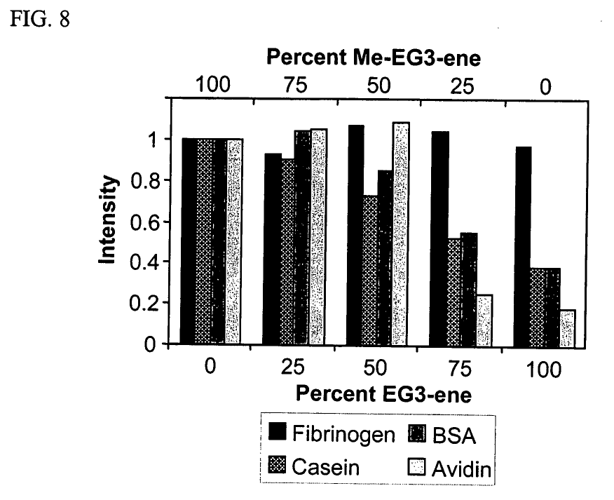

This part of the example demonstrates how the nature of the terminal group on the EG chain can affect non-specific protein adsorption. Represented in FIG. 8 is the on-chip fluorescence intensity data of avidin, BSA, casein, and fibrinogen adsorbed to monolayers of varying composition of EG3-ene and Me-EG3-ene on silicon. The fluorescence intensity from BSA, casein, and avidin adsorbed to the hydroxyl-terminated EG3-functional monolayers is only 20-40% of that observed on the methyl-terminated Me-EG3-functional monolayers, indicating that the hydroxyl group is more effective that the methyl group in decreasing the amount of non-specific adsorption. However, the additional methyl group did not affect the amount of fibrinogen that adsorbed to the surfaces. These observations show that the hydroxyl-terminated EG3 functionality is generally more effective than the methyl-terminated Me-EG3 functionality at resisting non-specific adsorption, although the difference in effectivness may be protein-dependent. Given that hydroxyl-terminated EG-functional monolayers present surfaces that are resistant to adsorption of the widest variety of proteins, for many applications its use may be preferable to methyl-terminated EG-functional monolayers.

Fibrinogen, which shows no significant preference for hydroxyl-EG3 vs. methyl-EG3 functionalities, has been observed to adsorb to both hydrophilic and hydrophobic surfaces by others. These previous studies have attributed this observation to the existance of both hydrophobic and hydrophobic domains within fibrinogen, which allow it to interact with both types of surfaces. (See, for example, Schwendel, D.; Dahint, R.; Herrwerth, S.; Schloerholz, M.; Eck, W.; Grunze, M., Langmuir 2001, 17, 5717-5720; Kim, J.; Somorjai, G. A., J. Am. Chem. Soc. 2003, 125, 3150-3158.) The unique elongated structure of fibrinogen (Fuss, C.; Palmaz, J. C.; Sprague, E. A., J. Vasc. Interv. Radiol. 2001, 12, 677-682) likely contributes to orientation-dependent changes in fibrinogen packing, as these physical packing forces may dominate the adsorption dynamics thereby weakening the effect of surface termination. For comparison, BSA contains hydrophobic pockets on its surface for the purpose of carrying fatty acid chains and is more globular in form. This suggests that BSA may be more affected by surface termination, associating more strongly with a surface that is more hydrophobic, as the Me-EG3 surface is.

Comparative Elution Measurements on Different Surfaces

While the above studies provide good qualitative insights into how the monolayers affect non-specific adsorption, on-chip fluorescence measurements cannot be easily used for absolute, quantitative analysis or even comparisons between different substrates (i.e., gold, Si, and diamond) because of the unknown amount of fluorescence quenching. To provide quantitative information on the extent of non-specific adsorption, a solution-based fluorescence method was used, wherein the proteins adsorbed to the surfaces were eluted into a known volume of solution, and the fluorescence intensity of the solution was then measured. A more detailed description of this method may be found in Enderlein, J., Biophysical Journal 2000, 78, 2151-2158, the entire disclosure of which is incorporated herein by reference. Stringent elution conditions under which the fluorescence intensity of the substrate was reduced by approximately 99% or more were used, indicating that more than 99% of the adsorbed protein was eluted into solution. The concentration of avidin in the eluted solution was calculated by comparing the fluorescence intensity of the eluted protein solution to a calibration curve (made from standards of known avidin concentration). The avidin calibration curve showed a linear dependence of fluorescence emission with concentration, and a detection limit of approximately 1.4 pgram/mL or 2.2 fmol/mL avidin.

To establish a baseline corresponding to a full “monolayer” of avidin, this method was first applied to surfaces that were modified with biotin, which binds strongly to avidin and is expected to produce a densely-packed layer of avidin molecules. As shown in FIG. 9, silicon and diamond surfaces were first amino-terminated, then biotinylated with a linker containing a disulfide bond, and finally exposed to fluorescein-labeled avidin. Avidin that bound to the surfaces was then eluted off by cleaving the disulfide bond in the biotin linker using mercaptoethanol in the elution buffer. Shown in FIG. 10 is the amount of avidin bound to the surfaces. Biotinylated gold bound 6.9 pmol/cm², silicon bound 4.9 pmol/cm², and diamond bound 7.7 pmol/cm² of avidin. As a point of comparison, the percent monolayer equivalent (% ML equ.) of a close-packed layer can be estimated using the molecular dimensions of avidin (40 Å×50 Å×56 Å), as described in amount of avidin bound to the surface was based on a molecular weight of 62,400 Da. Percent monolayer equivalent was calculated from the size of avidin (5.6 nm×5.0 nm×4.0 nm). A complete monolayer of avidin is between 3.6×10¹¹ molecules/cm² and 5.0×10¹¹ molecules/cm² (or 6.0 pmol/cm² and 8.3 pmol/cm²). Using this assumption, the results show that biotinylated gold binds 83% of a close-packed monolayer of avidin, silicon binds 60% of a monolayer, and diamond binds 93% of a monolayer.

All three surfaces bind less than what would be expected for a close-packed layer, and the three starting surfaces bind different amounts of avidin. While a full monolayer would correspond to 8.3 pmol/cm², steric-hindrance between avidin molecules and random adsorption (not close-packing) would likely prevent a 100% monolayer from forming on any surface. The diamond surface may have bound slightly more avidin than one would expect because the surface of NC diamond is rough due to the strong tetrahedral bonding and crystallite size of 200-500 nm. Comparing these results to other data in the literature, it has been reported that I¹²⁵ labeled avidin immobilized on a biotinylated Teflon surface bound approximately 5.4 pmol/cm² or 66% of a monolayer, (see McFarland, C. D.; Jenkins, M.; Griesser, H. J.; Chatelier, R. C.; Steele, J. G.; Underwood, P. A., J. Biomater. Sci. Polymer Edn 1998, 9, 1207-1225) which falls within the range of these data (between 60% and 93% of a monolayer). The results from these measurements and good correspondence with previous results from radioactive methods provides confidence that the use of elution combined with solution-based fluorescence measurements is a highly sensitive, accurate method for quantitatively analyzing avidin adsorption, and, by avoiding the well-known problems associated with quenching of molecules at surface, is a good way of quantitatively comparing different surfaces.

After ensuring that the elution buffer and fluorometer measurements yielded accurate results on biotinylated silicon, NC diamond, and gold, the effect of different surface terminations on non-specific protein adsorption was studied. Depicted in FIG. 10 are the results of elution experiments, in which avidin was exposed to surfaces with different terminations and then eluted off overnight. These data are plotted on a log scale of % ML equ. versus substrate type (NC diamond, silicon, and gold) and as a function of surface termination. To measure specific binding of avidin, the surface was biotinylated, whereas non-specific adsorption of avidin was measured on amino-, EG3-, or EG6-terminated monolayers, and the results are graphed in FIG. 10. The data show that for silicon and diamond, functionalization with the amino group reduces the amount of non-specific adsorption by approximately a factor of ten compared with the biotinylated surfaces (i.e., full monolayer), while amino-termination of gold reduced the non-specific adsorption by a factor of 2. For all three surfaces, modification with EG3 further reduced the amount of avidin adsorbed to them. Gold and NC diamond adsorbed approximately 3% ML equ. (0.24 pmol/cm²) avidin, while silicon adsorbed less, ˜1% ML equ. (0.074 pmol/cm²). Silicon and diamond were also functionalized with EG6 (EG6-termination on gold was not studied) and the data show that this yields a further reduction in the amount of adsorbed avidin, to 2% ML equ. or 0.16 pmol/cm² (diamond) and 0.7% ML equ. or 0.056 pmol/cm² (silicon).

These experiments demonstrate several important points. First, the data show that modification with EG3-terminated monolayers very effectively reduces non-specific protein adsorption on silicon, diamond, and gold surfaces. A comparison of the surfaces shows that EG3-modified diamond surfaces resist non-specific adsorption as effectively as EG3 SAMs on gold, and that EG3-modified silicon samples are the most effective of all. Finally, the data show that while EG3 functionality is effective at reducing non-specific adsorption of avidin, further reduction may be obtained by using longer EG chains.

Characterization of Monolayers

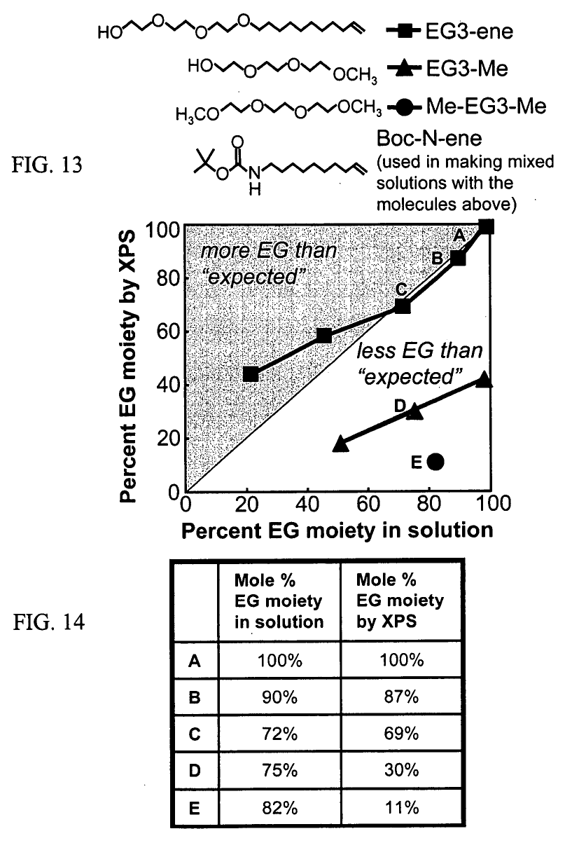

This part of the example describes a series of studies in which the compositions of surface monolayers produced by mixing various molecules with Boc-N-ene in varying mole fractions were measured, and the resulting surface compositions were analyzed using XPS. FIG. 13 graphically summarizes the composition of the surface monolayers as determined by XPS for various parent compositions, while FIG. 14 gives some specific values of surface composition. The labels, “A”, “B”, etc. in each part of this figure are consistent. To identify the molecules bound to the surface, use was made of the fact that in the EG molecules, the carbon atoms directly bound to oxygen atoms are shifted to a relatively high binding energy of 287.3 eV, (see e.g., Harder, P.; Grunze, M.; Dahint, R.; Whitesides, G. M.; Laibinis, P. E., J. Phys. Chem. B 1998, 102, 426-436; Pale-Grosdemange, C.; Simon, E. S.; Prime, K. L.; Whitesides, G. M., J. Am. Chem. Soc. 1991, 113, 12-20; Huang, N.-P.; Michel, R.; Voros, J.; Textor, M.; Hofer, R.; Rossi, A.; Elbert, D. L.; Hubbell, J. A.; Spencer, N. D., Langmuir 2001, 17, 489-498) giving rise to the peak at this energy that can be observed in the C(I s) spectra. The carbon atoms in the hydrocarbon chain appear at a lower binding energy of 285.8 eV. (See e.g., Harder, P.; Grunze, M.; Dahint, R.; Whitesides, G. M.; Laibinis, P. E., J. Phys. Chem. B 1998, 102, 426-436; Pale-Grosdemange, C.; Simon, E. S.; Prime, K. L.; Whitesides, G. M., J. Am. Chem. Soc. 1991, 113, 12-20; Huang, N.-P.; Michel, R.; Voros, J.; Textor, M.; Hofer, R.; Rossi, A.; Elbert, D. L.; Hubbell, J. A.; Spencer, N. D., Langmuir 2001, 17, 489-498.) (The t-Boc group of Boc-N-ene was removed under deprotection conditions prior to XPS characterization.) Thus, measuring the areas of these peaks and correcting for the known number of carbon atoms of each type in the parent molecules allows for the determination the surface composition. The percent EG moiety was calculated from XPS data using the following equation: X=% EG moiety, 100−X=% Boc-N-ene (100−X)/(X)=[(low BE Carbon area)/(high BE Carbon area−Nitrogen area)]*(# C having high BE)/(# C having low BE). The nitrogen area was corrected for the sensitivity factor difference between nitrogen and carbon.

The composition of mixed monolayers of EG3-ene and Boc-N-ene are addressed first. The square data points in FIG. 13 show the resulting surface compositions for five different solution compositions. These data show that when the mole percentage of EG3-ene in the parent solution is greater than 70%, the mole percentage on the surface accurately reflects the parent solution composition (points C-A in FIG. 13). However, when the parent solution contained less than 70% EG3-ene (and therefore more than 30% Boc-N-ene), the surface showed a higher EG3 concentration than the parent solution did, as demonstrated by the points that lie in the “more than expected” region of FIG. 13. This deviation may be attributed to steric hindrance between the bulky Boc groups, which allow the smaller EG3 molecules to more effectively pack between the Boc-N-ene molecules and thereby increase the amount of EG3 relative to Boc-N-ene on the surface.

Optimization for Biosensing

A common geometry for surface-based biosensors is to immobilize a given probe molecule on the surface and detect a given target molecule in solution. In this part of the example, the optimum density of probe molecule on the surface that gives the highest ratio of specifically captured target to non-specifically adsorbed target molecule was investigated. In addition the possibility of detecting a given target molecule within a solution that contains many different types of molecules was examined. These studies were conducted using mixed monolayers of EG6-ene and biotin, the model probe molecule, on silicon and exposing the surface to avidin, the model target molecule. Chicken serum was used as a background matrix.

The optimum density of probe molecules was explored by forming mixed amino- and EG6-terminated monolayers on silicon. To evaluate specific binding and non-specific adsorption in a single experiment, the entire surface was functionalized with a mixture of EG6-ene and Boc-N-ene that was subsequently deprotected to produce a mixed monolayer consisting of amino groups separated by EG6 molecules. Using a microfluidic circuit, the terminal amino groups in some locations were then reacted with a biotin linker, while the monolayer on the rest of the surface was left alone. This process produces a mixed monolayer that is comprised of molecules that resist non-specific adsorption (EG6-terminated oligomers) mixed with a controlled number of embedded biotin molecules that act as sites for specific binding of avidin, as shown in FIGS. 11a and 11b. Surfaces functionalized with varying densities of biotin were then exposed to a 20 μg/mL fluorescent-avidin solution, and the adsorption of avidin was then characterized by on-chip fluorescence imaging; the intensity of fluorescence in the biotinylated regions was attributed to specific binding, while that in to the non-biotinylated region was attributed to non-specific adsorption. The overall quality of the surface can be parameterized by the ratio of specifically bound avidin to non-specifically adsorbed avidin, which is defined as the SINS ratio.

When no EG6-termination was present in the monolayer, the fluorescence intensity was high on the regions that were biotin modified, but the SINS ratio in FIG. 11a was low. However, in FIG. 11b, the percentage of EG6-ene in the parent solution was increased to 90% (10% Boc-N-ene), which improved the contrast of the fluorescence image dramatically. The graph in FIG. 12 shows the substantial increase in the SINS ratio by incorporating EG units into monolayers. In the case of the EG6-terminated monolayer, the optimum parent solution composition (90% EG6-ene and 10% amino) resulted in a factor of 19 improvement over the 100% amino monolayer (22.8/1.21). A maximum occurred at 10% amino, 90% EG6-ene because the intensity of the specifically bound avidin was almost equal to the intensity on a 100% amino surface (controlled by steric effects from adjacent avidin molecules), and the amount of non-specifically adsorbed avidin was dramatically reduced. These results as well as the results on EG3-ene modified silicon from Example 1 are presented in the graph in FIG. 12. The maximum SINS ratio when using EG3-terminated monolayers was 9.10, but use of EG6 termination instead of EG3 in the monolayer increased the SINS ratio by a factor of 2.

It should be noted that the x-axis in FIG. 12, is the percent amino that existed in the parent solution, not the percent amino that actually attached to the surface, -and as discussed above, these values can vary significantly. XPS characterization of EG3-functional mixed monolayers showed that at 70% or more EG3-ene in the parent solution resulted in the same percentage of EG3-termination on the surface. However, in the case of EG6-terminated monolayers, this rule does not hold. A mixed monolayer made from a parent solution of 90% EG6-ene and 10% Boc-N-ene resulted in a surface composition of 69% EG6-termination and 31% amino-termination by XPS (data not shown), the same optimum surface composition found when using EG3-ene. These data demonstrate that functionalized surfaces composed of approximately 70% EG(3 or 6)-termination and 30% amino-termiantion resulted in a maximum SINS ratio of specifically bound to non-specifically adsorbed avidin.

Since biosensing assays typically involve detection of one component within complex mixtures of many components, the selectivity of functionalized silicon surfaces was tested by exposing both biotinylated monolayers and biotin embedded within EG6-functional monolayers to chicken serum, a complex mixture of proteins, to which fluorescent avidin was added. Biotin-modified silicon surfaces were prepared from 100% Boc-N-ene (FIG. 11a) and from 90% EG6-ene, 10% Boc-N-ene (FIG. 11b) which were then biotinylated with an amine-reactive biotin linker. Chicken serum was spiked with fluorescein-labeled avidin to make serum solutions having avidin concentrations between 20 μg/mL and 0.2 [μg/mL. The biotin-modified silicon samples were then immersed in the avidin/serum solutions for 1 hr. The fluorescence intensity was measured in two places on each sample: on the biotinylated stripe (which specifically bound avidin) and on the surrounding area (to which avidin non-specifically adsorbed). Because the composition of the monolayer was constant for each data set, the non-specifically adsorbed fluorescent-avidin (NS) was subtracted from the specifically bound fluorescent-avidin (S) and the data plotted as shown in FIG. 15. The fluorescence intensity of the biotinylated silicon surfaces that had been functionalized with 90% EG6-ene/10% Boc-N-ene was almost twice as high as the biotinylated 100% Boc-N-ene surfaces. This difference indicates that significantly more avidin was able to bind to biotin molecules immobilized on EG6 regions than on the amino regions. And, we attribute the difference in the intensities of the two types of functionalized surfaces to the non-specific adsorption of serum proteins which block fluorescein-avidin from binding biotin on the biotinylated 100% amino surface more than on the biotinylated EG6 surface. The detection limit of this assay was approximately 3 nM avidin, which is likely limited due to mass transport phenomena.

These results demonstrate that EG-containing monolayers may be used to improve two parameters in biosensors. First, the SINS ratio may be increased by reducing non-specific absorption. And second, the selectivity of monolayers containing EG6 can be enhanced to bind a specific protein while resisting the non-specific adsorption of others, although the detection limit is not controlled by non-specific protein adsorption.

It is understood that the invention is not confined to the particular embodiments set forth herein, but embraces all such forms thereof as come within the scope of the following claims.

What is claimed is:

1. A surface-modified substrate comprising:
a. a silicon or carbon substrate having a surface; and
b. a layer comprising hydroxyl-terminated ethylene glycol oligomers covalently bound to the surface.

2. The substrate of claim 1, wherein the ethylene glycol oligomers have the formula CH₂═CH(CH₂)ₘ(OCH₂CH₂)ₙOH, where m>0 and 3≦n≧20.

3. The substrate of claim 1, wherein the ethylene glycol oligomers have the formula CH₂═CH(CH₂)ₘ(OCH₂CH₂)ₙOH, where m>0 and 3≦n≧9.

4. The substrate of claim 1, wherein the substrate is a silicon substrate.

5. The substrate of claim 4, wherein the substrate is a single crystal silicon substrate and the surface is a Si(111) surface.

6. The substrate of claim 1, wherein the substrate is a carbon substrate.

7. The substrate of claim 6, wherein the substrate is selected from the group consisting of diamond substrates, glassy carbon substrates, diamond-like carbon substrates, graphitic carbon substrates and pyrolytic carbon substrates.

8. The substrate of claim 1, wherein the layer comprises a monolayer.

9. The substrate of claim 1, wherein the layer further comprises probe molecules covalently bound to the surface.

10. The substrate of claim 1, wherein the substrate comprises a medical implant.

11. A sensor device comprising:
a. a silicon or carbon substrate having a surface; and
b. a layer of molecules covalently bound to the surface, the layer at least partially comprising a random distribution of ethylene glycol oligomers and probe molecules.

12. The device of claim 11, wherein the ethylene glycol oligomers have the formula CH₂═CH(CH₂)ₘ(OCH₂CH₂)ₙOH, where m>0 and 3≦n≧20.

13. The device of claim 11, wherein the ethylene glycol oligomers have the formula CH₂═CH(CH₂)ₘ(OCH₂CH₂)ₙOH, where m>0 and 3≦n≧9.

14. The device of claim 11, wherein the substrate is a silicon substrate.

15. The device of claim 14, wherein the substrate is a single crystal silicon substrate and the surface is a Si(111) surface.

16. The device of claim 11, wherein the substrate is a carbon substrate.

17. The device of claim 16, wherein the substrate is selected from the group consisting of diamond substrates, glassy carbon substrates, diamond-like carbon substrates, graphitic carbon substrates and pyrolytic carbon substrates.

18. The device of claim 11, wherein the layer comprises a monolayer.

19. The device of claim 11, wherein the random distribution comprises about 60 to 80% ethylene glycol oligomers and about 20 to 40% probe molecules.

20. The device of claim 11, wherein the random distribution comprises about 65 to 75% ethylene glycol oligomers and about 25 to 35% probe molecules.

21. The device of claim 11, wherein the probe molecules comprise biomolecules.

22. The device of claim 21, wherein the biomolecules comprise proteins.

23. The device of claim 22, wherein the proteins comprise biotin molecules.

24. The device of claim 21, wherein the biomolecules are selected from the group consisting of DNA molecules, RNA molecules, oligonucleotides, peptides, polypeptides, proteins, enzymes, antibodies, receptors, polysaccharides, viruses and combinations thereof.

25. A method of detecting target molecules in a sample, the method comprising exposing the sample to the sensor device of claim 11, wherein the sample contains molecules capable of undergoing specific binding interactions with the probe molecules.

26. A surface-modified substrate comprising:
a. a carbon substrate having a surface; and
b. a layer comprising ethylene glycol oligomers covalently bound to the surface.

27. The substrate of claim 26, wherein the ethylene glycol oligomers have the formula CH₂═CH(CH₂)ₘ(OCH₂CH₂)ₙOR, where m>0 and 3≦n≧20 and R is an atom or functional group selected from the group consisting of H atoms, methyl groups, amino groups and carboxyl groups.

28. The substrate of claim 26, wherein the ethylene glycol oligomers have the formula CH₂═CH(CH₂)ₘ(OCH₂CH₂)ₙOR, where m>0 and 3≦n≧9 and R is an atom or functional group selected from the group consisting of H atoms, methyl groups, amino groups and carboxyl groups.

29. The substrate of claim 26, wherein the substrate is selected from the group consisting of diamond substrates, glassy carbon substrates, diamond-like carbon substrates, graphitic carbon substrates and pyrolytic carbon substrates.

30. The substrate of claim 26, wherein the layer comprises a monolayer.

31. The substrate of claim 26, wherein the substrate comprises a medical implant.
